(19) United States
(12) Patent Application Publication
(10) Pub. No.: US 2003/0028214 A1
(43) Pub. Date: Feb. 6, 2003

(54) Vascular compression apparatus

(76) Inventors: Philip David Benz, Tigard, OR (US); Herbert J. Semler, Portland, OR (US); Benjamin Peter Mergen, Corbett, OR (US); William Richard Huseby, Vancouver, WA (US)

Correspondence Address: Philip D. Benz, 2852 SW 64th Place, Portland, OR, 97221, US

(21) Appl. No.: 09/923,680

(22) Filed: Aug. 6, 2001

(51) Int. Cl. A61B 17/00

(52) U.S. Cl. 606/201

(57) ABSTRACT

A vascular compression apparatus and method for applying pressure onto an area of a patient generally including a blood vessel and a wound site, such as a blood vessel puncture, after a cannulated procedure for the purpose of controlling bleeding and achieving hemostasis. The vascular compression apparatus includes a handle, an elongate shaft and a pad. The shaft extends generally downward from the cente of the bottom side of the handle, The pad is connected generally at the center of its top side to the bottom end of the shaft. In use, the pad is generally placed proximal to the catheter insertion site and over the blood vessel containing the catheter. The catheter is then removed from the blood vessel and pressure applied to the gandle by the user in a downward direction to force the pad to compress the blood vessel for the purpode of controlling bleeding and, further, to achieve hemostasis. The entire vascular compression apparatus may then be discarded after use.

### CROSS-REFERENCE TO RELATED APPLICATIONS

Not applicable.

### STATEMENT REGARDING FEDERALLY SPONSORED RESEARCH OR DEVELOPMENT

Not applicable.

### REFERENCE TO A MICROFICHE APPENDIX

Not applicable.

### BACKGROUND OF THE INVENTION

This invention relates to a vascular compression apparatus and method for controlling bleeding and achieving hemostasis by applying pressure onto an area of a patient including a wound site, such as a blood vessel puncture, and a blood vessel. In particular, this invention relates to a vascular compression apparatus and a method for controlling bleeding and achieving hemostasis in the femoral area of a groin following completion of a procedure which involves cannulation of a femoral artery for the purpose of diagnostic catheterization or angioplasty.

The femoral artery is a high pressure blood vessel which requires deliberate action to achieve hemostasis (cessation of bleeding) following completion of a cannulation procedure. If a sheath is removed from, for example, the femoral artery in the groin and no attempt to achieve hemostasis is made, the patient would quickly experience rapid bleeding resulting in adverse health consequences including hypovolemia, shock, a requirement for blood transfusion, and possibly death. Application of pressure proximal and medial to a femoral artery puncture site and directly over the femoral artery is a means of achieving hemostasis, where such pressure is sufficient to slow or completely occlude blood flow in the femoral artery. This permits a clot to form which causes hemostasis at the puncture site.

One means of applying such pressure requires that an individual remain with the patient, actively pressing down with gloved hands directly over the artery and proximal and medial to the puncture site for a period of time which varies based on the type of procedure, the nature of the drugs administered and the patient's condition; frequently this period of time extends between 15 and 30 minutes or longer. As a result, fatigue, stiffness and pain may occur in the fingers, hands, wrist and forearms of the practitioner performing this procedure. Also, there is the possibility that a glove could have or develop a hole, thereby allowing direct pressurized skin contact with a patient's blood. An individual repeatedly performing this procedure over a long period of time without the aid of any assist devices could develop repetitive strain injury, for example, carpal tunnel syndrome.

One possible solution for controlling blood flow and achieving hemostasis is described by Nobis in U.S. Pat. No. 3,411,505. This device has a handle at one end and a rod at its other end which is inserted through an incision in the body surface and directly contacts arteries to be compressed. A drawback to this device is that it is hard to balance on the artery when supported by hand and may tend to slip or fall over.

Another important drawback is that the device was specifically not designed for external use and is therefore not suitable for applying pressure onto the body surface.

Another possible solution is described by Semler in U.S. Pat. No. 3,779,249 and U.S. Pat. No. 5,304,186. The devices described in these patents are generally comprised of a c-clamp mechanism, to which a removable, disposable, sterile pad is attached when being used on a patient. An advantage of using the c-clamp mechanism is its ability to free the hands of the attending practitioner. A drawback of this device is the cost associated with the c-clamp mechanism. Another drawback is the inconvenience of locating the apparatus, ensuring a minimal level of cleanliness, and connecting to it the removable pad prior to its use on a patient. Further, following use, the c-clamp must be removed from the patient area and cleaned and stored, and the removable pad detached and disposed of. A disposable sterile pad, which can be used with the c-clamp device, is described by Royse in U.S. Pat. No. 4,572,182.

Another possible solution is an apparatus which is described by Toller in U.S. Pat. No. 5,342,388. Said apparatus is comprised of an elongated substantially cylindrically shaped handle weighing at least about one pound, an elongated rod extending outward from said handle, to which a removable, disposable, sterile disk is attached when being used on a patient. A disadvantage of this apparatus is the substantial cost to the user associated with obtaining the apparatus. Another disadvantage is the inconvenience of locating the apparatus, ensuring a minimal level of cleanliness, and connecting to it the removable disk prior to its use on a patient. Further, following use, the apparatus must be removed from the patient area and cleaned and stored, and the removable pad detached and disposed of. A disposable sterile disk, which can be used with said apparatus, is described by Royse in U.S. Pat. No. 4,572,182.

### SUMMARY OF THE INVENTION

An object of this invention is to provide an improved apparatus to assist a user in controlling bleeding and achieving hemostasis, through the application of pressure onto a body surface thereby compressing the lumen of an underlying blood vessel. This compression in turn reduces or halts blood flow through said blood vessel.

An additional object of the invention is to assist a user in controlling bleeding and achieving hemostasis with a hand held device without requiring excessive hand, finger, or wrist exertion by the user.

Another object of the invention is to increase convenience to the user by providing all elements required to achieve hemostasis as a single, unitary member, thereby eliminating required assembly of the apparatus by the user prior to use.

A further object of the invention is to assist a user in controlling bleeding and achieving hemostasis after removing a catheter or cannula from a blood vessel and to achieve hemostasis at the puncture site using a device which may be disposed of in its entirety following completion of its use.

A further object of the invention is to reduce the cost associated with equipment required to achieve hemostasis.

These and other objects are achieved with the vascular compression apparatus described herein. The vascular compression apparatus is comprised of a handle, an elongate shaft, and a pad. The proximal end of said shaft connects to said handle generally in the center of the bottom side of the handle. Said pad connects to the distal end of the shaft generally in the center of the top side of the pad. The pad is generally shaped to rest on the body of a patient such that when downward pressure is applied by a user onto the handle, the pad depresses the body surface upon which it rests, thereby compressing the lumen of the blood vessel over which it is placed to limit or completely stop the flow of blood during such application of pressure.

The handle may be in the shape of a dome having an open bottom side, generally in the center of which the shaft connects. The dome-shaped handle may have a generally circular or generally oval perimeter.

The pad may be round, formed as a disk with an upturned perimeter and a v-shaped notch cut into its perimeter, similar to the pad described by Royse in U.S. Pat. No. 4,572,182 which is herein incorporated by reference. The pad may alternatively take another shape which accommodates similar function; for example, the pad may be generally formed in the shape of three fingers held together side by side.

An important advantage of the v-shaped notch is that it enables proper placement of the vascular compression apparatus onto the body surface in proximity to a puncture site, and overlies the blood vessel for which compression is desired. In addition, it permits visualization of and access to the puncture site while compression is being applied.

Portions of the surface of the apparatus may have a surface treatment, material or coating which modifies the coefficient of friction of such surfaces, minimizing the opportunity for slipping along the surface of the patient's body or slipping in the user's hand.

In one embodiment, the handle and elongate shaft of the vascular compression apparatus are formed as a single member and the pad is removably connected to the elongate shaft. In a preferred embodiment, the vascular compression apparatus is formed as a unitary member with the pad permanently connected to the elongate shaft, thereby requiring no assembly or disassembly by the user.

The vascular compression apparatus therefore achieves its objects: the apparatus helps a user control blood flow and achieve hemostasis, and reduces or eliminates excessive hand, finger or wrist exertion; cost of equipment is reduced, in that no other equipment is required; user convenience is increased since there are no moving parts or adjustments, assembly and disassembly by the user is not required, and the entire device may be disposed of in its entirety following use.

In particular the vascular compression apparatus achieves the following benefits, relative to the prior art:

(a) Reduced fatigue, strain and risk of disability result from the shape of the handle, which enables the user to position their upper arm, elbow, forearm, wrist and hand such that significant exertion by muscles in these body parts is minimized; in use, a user (i) places the palm of their hand directly over the topmost area of the handle and bends their wrist so that the palm of their hand faces downward and covers a portion of the top surface of the handle, and (ii) keeping their elbow straight, comfortably applies pressure downwards onto the handle with the base of the palm, without significant exertion of muscles in the forearm, wrist or hand; this shape additionally permits other methods of easily exerting downward pressure onto the handle enabling different postures and muscles to be used in such pressure application, including simultaneous use of two hands, to avoid fatiguing or straining the same set of muscles through repetitive use;

(b) Increased convenience and ease of use results from the unitary construction of the preferred embodiment of the vascular compression apparatus since no additional items need to be located, and assembly or cleaning is not required prior to use; in addition, following use, cleaning or disassembly is not required and the entire apparatus may be disposed of in its entirety;

(c) Lower cost results from providing a single apparatus composed of inexpensive common materials, for example an acrylic or a plastic, and which does not require any additional equipment in order to use it;

(d) Puncture site complications may be avoided as a result of the surface treatment, material or coating, which may be applied to the bottom surface of the pad for the purpose of eliminating or greatly reducing any slippage of the pad across the body surface during application of the vascular compression apparatus; such slippage, if it occurs, can cause post-catheterization bleeding complications such as hematoma or pseudoaneurysm. dr

### BRIEF DESCRIPTION OF THE DRAWINGS

FIG. 1 is a perspective view of the invention;

FIG. 2 is a side sectioned view of the invention shown in FIG. 1;

FIG. 3 is a top view of a handle of the invention;

FIG. 4 is another top view of an alternate shape of the handle of the invention;

FIG. 5 is a partial side sectioned view of the invention closing a blood vessel;

FIG. 6 is a perspective view of a pad of the invention;

FIG. 7 is a side sectioned view of the pad shown in FIG. 6;

FIG. 8 is a side sectioned view of an alternative configuration of the invention;

FIG. 9 is a side sectioned view of an alternative configuration of the invention featuring a pivoting means.

### DETAILED DESCRIPTION OF THE INVENTION

The vascular compression apparatus is used on a patient to apply pressure on an area near a wound site, such as a blood vessel puncture, after a cannulated procedure such as angioplasty, for the purpose of controlling the patient's bleeding and, further, of achieving hemostasis.

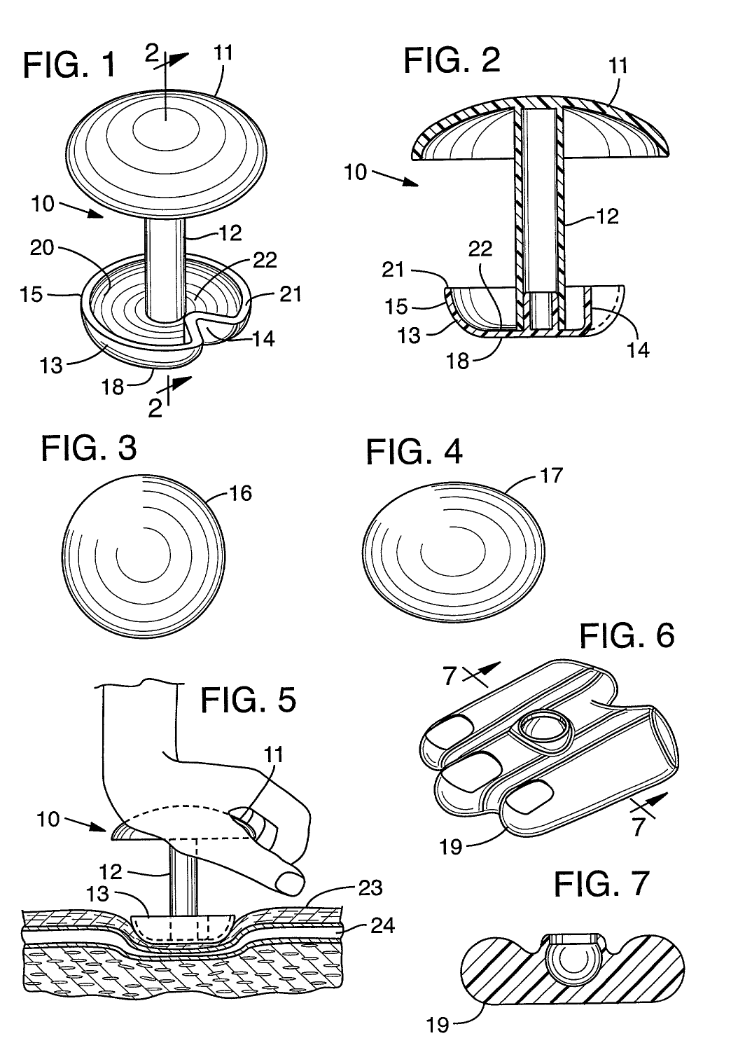

Referring to FIGS. 1-9 there is shown an invention 10.

FIG. 1 shows the handle 11 connected to the proximal end of the shaft 12. The pad 13 is connected to the distal end of the shaft 12 generally in the center of the top surface 22 of the pad 13. The handle 11 is generally dome-shaped and includes a substantially hollow bottom side.

FIG. 1 further shows the pad 13 in the general shape of a round disk with a v-shaped notch 14, generally disclosed in U.S. Pat. No. 4,572,182 which is herein incorporated by reference. The v-shaped notch 14 has its greatest width at the perimeter of the pad, and its apex pointing generally towards the center of the pad. Further, a peripheral area of the pad 13 may be upwardly curved so that the rim 21 of the disk is at a level higher than that of the flat portion of the top surface 22 of the disk. This peripheral area may include an upturned perimeter 15, a radius 20 at the point at which the upturned perimeter 15 begins to curve upward, and the rim 21. The pad 13 has a bottom surface 18.

FIG. 2 shows a side sectioned view of the invention 10.

FIG. 3 shows a top view of the handle 16, the handle having a generally circular perimeter. FIG. 4 shows a top view of an alternative shape of the handle 17, this shape having a generally oval perimeter.

FIG. 5 shows a partial side sectioned view showing application of the vascuiar compression apparatus 10 to a patient, whereby the pad 13 is placed on the patient's body surface 23 generally over the blood vessel 24 for which control of bleeding and hemostasis is desired. When downward pressure is applied by a user onto the handle 11 such pressure is transferred through the shaft 12 to the pad 13. The pad 13 depresses the area of the body surface 23 upon which it rests, thereby compressing the lumen of the blood vessel 24 over which it is placed to partially or completely occlude the blood vessel. Such occlusion limits or completely stops the flow of blood in the blood vessel 24 during such application of pressure, enabling hemostasis to occur. Once hemostasis occurs the vascular compression apparatus 10 is removed from the patient and discarded in its entirety. It can be appreciated that the vascular compression apparatus 10 can also be used to externally compress other luminal tissue for the purpose of controlling fluid flow therein.

FIG. 6 shows a perspective view of an alternative shape of a pad 19, in the form of three fingers generally positioned together side-by-side, which may be incorporated into an alternative embodiment of the invention.

FIG. 7 shows a side sectioned front view of the pad 19.

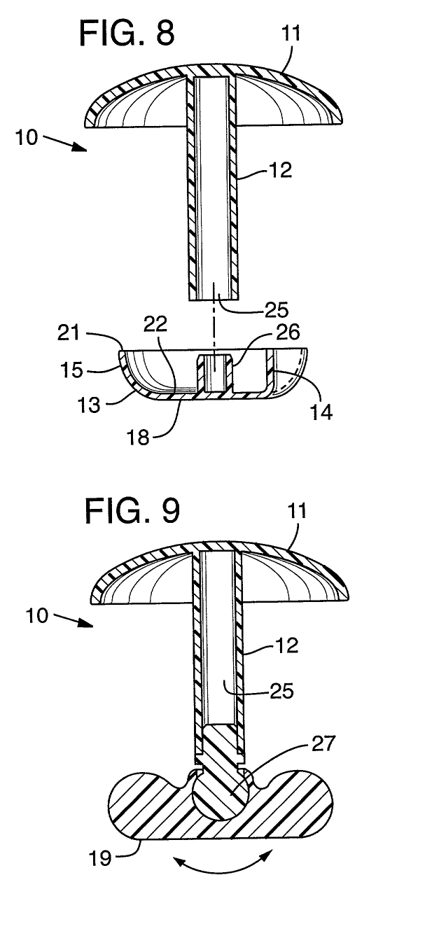

FIG. 8 shows a side sectioned view of an alternative configuration of the invention, where the handle 11 and shaft 12 are composed as a single element. The pad 13 is removable from the shaft 12. The shaft 12 includes a lumen 25 extending longitudinally therethrough to the distal end of the shaft 12. The pad 13 includes a cylindrical member 26 protruding upwards and located generally in the center of the flat top surface 22 of the pad. Connection of the pad 13 to the shaft 12 is achieved by sliding the cylindrical member 26 of the pad into the lumen 25, whereby the outer surface of the cylindrical member 26 is in contact with the surface of the lumen 25 such that the pad 13 is detachably held therein by friction.

FIG. 9 shows a side sectioned view of another alternative configuration of the invention, where a pivoting means 27 connects the shaft 12 to the pad 19. The pad 19 is attached by a pivoting means 27 whereby the pad may angulate or rotate about its axis. Such attachment may permit detachment and reattachment, so that the pad 19 is a member separate from the member comprising the shaft 12 and the handle 11. Such pivoting means may be in the form of a ball-and-socket joint or a modification thereof.

Molded of a generally rigid material, for example, an acrylic or a plastic, the vascular compression apparatus 10 is sturdy enough to withstand the application of downward pressure onto a human patient, sufficient to cause a complete occlusion of an artery. The vascular compression apparatus 10 may be packaged and sterilized as a sterile medical product so that the user need not clean or wash it prior to its use.

The handle 11, in a preferred embodiment, is generally shaped as a dome, having an open underside, and a generally corstant dimension between the top and bottom surfaces of the handle 11. The handle 11 may have either a substantially circular 16 or substantially oval 17 perimeter. This shape enables a user to place the base of the palm of their hand directly over the topmost area of the handle 11 and, bending their wrist so that the palm of their hand faces downward and keeping their elbow straight, comfortably apply pressure downwards without significant exertion of muscles in the forearm, wrist or hand while maintaining a stable attitude of the vascular compression apparatus 10. This shape additionally permits other methods of easily exerting downward pressure onto the handle enabling different muscles to be used in such pressure application, including simultaneous use of two hands. Therefore, this general dome shape helps to significantly reduce fatigue and strain on the muscles and joints of the user's forearm, wrist and hand, helping to avoid potential repetitive strain injury. The open underside of the handle 11 also enables easy manipulation of the vascular compression apparatus.

In use, the pad 13 is generally placed proximal to the catheter insertion site and over the blood vessel 24 containing the catheter. The catheter or cannula is then removed from the blood vessel 24 and pressure applied to the handle 11 by the user in a downward direction to force the pad 13 to compress the blood vessel 24 for the purpose of controlling bleeding and, farther, to achieve hemostasis.

The proximal end of the shaft 12 connects to the handle 11 generally in the center of the hollow bottom side of the handle 11. In a preferred embodiment, the shaft 12 may have a generally circular section, or a circular section including ribs on either its interior or exterior surface. The length of the shaft 12 is at least sufficient to provide ample space for motion of the user's fingers when using the vascular compression apparatus 10 on a patient, but not so long that it inhibits the user's ability to maintain a generally straight elbow and stable attitude in the application of downward pressure.

In an alternative embodiment the pad 19 may farther include a connection to the shaft 12 comprising a pivoting means 27 which permits rotation and angulation to better enable conformance of the pad 19 to the contours of a patient's body surface 23.

In another alternative embodiment, certain portions of the vascular compression apparatus 10 may be treated or have applied to it a material to modify the coefficient of friction of the surfaces to which such treatment or material is applied. An application of this treatment or material has the effect of minimizing or eliminating slippage so that post-catheterization complications at the puncture site are avoided, where such treatment or material is applied to the bottom surface 18 of the pad 13. Such treatment or material may also prevent slippage of the vascular compression apparatus in the user's hand, also helping to prevent slippage or other undesired movement on the patient's body surface, where such treatment or material is applied to the top surface of the handle 11.

In another alternative embodiment, the composition of the vascular compression apparatus 10 may be changed to a heavier material, or material may be added to portions of the vascular compression apparatus to make it sufficiently heavy to achieve partial or total occlusion of a blood vessel 24 without the exertion of downward pressure on the handle 11.

In another alternative embodiment, the handle 11 and shaft 12 of the vascular compression apparatus 10 may be formed as a single member, to which the pad 13 may be removably connected by the user prior to use.

This detailed description of the invention is for illustrative purposes only. A reading by those skilled in the art will bring to mind various changes without departing from the spirit and scope of the invention.

We claim:

1. A vascular compression apparatus for applying pressure to tissue in proximity to a puncture site and a blood vessel; said vascular compression apparatus comprising as a unitary member:
(a) a handle;
(b) an elongated shaft extending generally perpendicularly from said handle; and
(c) a pad permanently connected to said elongated shaft at a distal end from the handle and generally adapted to rest on a body such that when generally downward pressure is applied to the handle, a lumen of said blood vessel is substantially compressed; said pad having a generally flat bottom side, of a certain size, which makes contact with the body surface.

2. The vascular compression apparatus as recited in claim 1 wherein said vascular compression apparatus has sufficient weight to generally close a femoral artery when said pad rests on said body surface and in the absence of downward pressure applied to said handle.

3. The vascular compression apparatus as recited in claim 2 wherein said sufficient weight being a removable and reusable member of said vascular compression apparatus.

4. The vascular compression apparatus as recited in claim 1 wherein said elongated shaft extends into said handle through a center of gravity of said handle.

5. The vascular compression apparatus as recited in claim 1 wherein said pad being formed as a disk having a substantially flat central top surface and including a generally upturned portion of said surface extending around the circumference of the pad; a v-shaped notch extending from the periphery of the pad towards a center portion of the pad, said v-shaped notch having its largest width at said periphery and its apex pointed generally toward the center of the pad; the pad having a substantially flat bottom side such that said vascular compression apparatus can maintain an upright position without external support when the pad rests on a flat surface.

6. The vascular compression apparatus as recited in claim 5 wherein portions of the surface of said vascular compression apparatus are modified by one or more means selected from the group comprising incorporation of a different material, overmolding using a different material, a surface treatment, a surface texture, and a coating; said portions of the surface thereby having a different coefficient of friction than the surfaces of other portions of the vascular compression apparatus.

7. The vascular compression apparatus as recited in claim 1 wherein said pad being formed as one or more fingers generally held together; the tips of said fingers being at one end, and a v-shaped notch being at the opposite end; said v-shaped notch having its largest width at the periphery of the pad and its apex pointed generally toward the center of the pad; the pad having a substantially flat bottom side such that the vascular compression apparatus can maintain an upright position without external support when the pad rests on a flat surface.

8. The vascular compression apparatus as recited in claim 7 wherein the connection between said pad and said elongated shaft includes a pivoting means permitting both a rotating and an angulating motion of the pad relative to the elongated shaft.

9. The vascular compression apparatus as recited in claim 7 wherein portions of the surface of said vascular compression apparatus are modified by one or more means selected from the group comprising incorporation of a different material, overmolding using a different material, a surface treatment, a surface texture, and a coating; said portions of the surface thereby having a different coefficient of friction than the surfaces of other portions of the vascular compression apparatus.

10. The vascular compression apparatus as recited in claim 1 wherein the handle is generally formed in a dome shape; the convex surface of said handle comprising the top surface of the handle; the concave surface of the handle comprising the bottom surface of the handle; the thickness of the handle between said top surface and said bottom surface being generally constant except for minor surface and structural features; said elongated shaft being connected to the handle generally in the center of the bottom surface of the handle.

11. The vascular compression apparatus as recited in claim 10, wherein said handle is generally formed in a dome shape having a substantially circular perimeter.

12. The vascular compression apparatus as recited in claim 10, wherein said handle is generally formed in a dome shape having a substantially oval perimeter.

13. A vascular compression apparatus for applying pressure to tissue in proximity to a puncture site and a blood vessel, wherein construction of said vascular compression apparatus includes the following steps:
(a) molding of a plurality of individual elements comprising the vascular compression apparatus; said individual elements generally including a handle, an elongated shaft extending generally perpendicularly from said handle, and a pad permanently connected to said elongated shaft at a distal end from the handle and generally adapted to rest on a body such that when generally downward pressure is applied to the handle, a lumen of said blood vessel is substantially compressed; said pad having a generally flat bottom side, of a certain size, which makes contact with the body surface.
(b) assembly of said individual elements into the vascular compression apparatus by a manufacturer of the vascular compression apparatus.

14. A vascular compression apparatus for applying pressure to tissue in proximity to a puncture site and a blood vessel; said vascular compression apparatus including a handle, an elongated shaft extending generally perpendicularly from said handle, and a pad removably connected to said elongated shaft at a distal end from the handle and generally adapted to rest on a body such that when generally downward pressure is applied to the handle, a lumen of said blood vessel is substantially compressed; said pad having a generally flat bottom side, of a certain size, which makes contact with the body surface.

15. The vascular compression apparatus as recited in claim 14, wherein said pad being formed as a disk having a substantially flat central top surface and including a generally upturned portion of said surface extending around the circumference of the pad; a v-shaped notch extending from the periphery of the pad towards a center portion of the pad, said v-shaped notch having its largest width at said periphery and its apex pointed generally toward the center of the pad; the pad having a substantially flat bottom side such that said vascular compression apparatus can maintain an upright position without external support when the pad rests on a flat surface.

16. The vascular compression apparatus as recited in claim 15 wherein portions of the surface of said vascular compression apparatus are modified by one or more means selected from the group comprising incorporation of a different material, overmolding using a different material, a surface treatment, a surface texture, and a coating; said portions of the surface thereby having a different coefficient of friction than the surfaces of other portions of the vascular compression apparatus.

17. The vascular compression apparatus as recited in claim 15 wherein said pad being formed as one or more fingers generally held together; the tips of said fingers being at one end, and a v-shaped notch being at the opposite end; said v-shaped notch having its largest width at the periphery of the pad and its apex pointed generally toward the center of the pad; the pad having a substantially flat bottom side such that the vascular compression apparatus can maintain an upright position without external support when the pad rests on a flat surface.

18. The vascular compression apparatus as recited in claim 17 wherein the connection between said pad and said shaft includes a pivoting means permitting both a rotating and an angulating motion of the pad relative to the shaft.

19. The vascular compression apparatus as recited in claim 17 wherein portions of the surface of said vascular compression apparatus are modified by one or more means selected from the group comprising incorporation of a different material, overmolding using a different material, a surface treatment, a surface texture, and a coating; said portions of the surface thereby having a different coefficient of friction than the surfaces of other portions of the vascular compression apparatus.
